(19) United States
(12) Patent Application Publication
(10) Pub. No.: US 2006/0275508 A1
(43) Pub. Date: Dec. 7, 2006

(54) Orally Administered Adsorbent Having Excellent Property for Adsorbing Nitrogen-Containing Compounds

(76) Inventors: Hideaki Kurosaki, Nigata-ke (JP); Masanori Tanaka, Nigata-ke (JP); Teiji Sato, Nigata-ke (JP); Kiyoshi Abe, Nigata-ke (JP); Toshio Ito, Tokyo (JP)

Correspondence Address: HAHN & VOIGHT PLLC, 1012 14TH STREET, NW, SUITE 620, WASHINGTON, DC, 20005, US

(21) Appl. No.: 11/424,174

(22) Filed: Jun. 14, 2006

(30) Foreign Application Priority Data
Aug. 27, 2004 (JP) 2004-248591
Aug. 19, 2005 (JP) 2005-238798

(51) Int. Cl. A61K 33/06

(52) U.S. Cl. 424/684

(57) ABSTRACT

An orally administered adsorbent which, when orally administered to livestocks and men, is capable of effectively adsorbing and removing nitrogen-containing compounds such as creatinine and the like. The orally administered adsorbent comprises clay particles of a layered structure having a cation-exchange capacity of not smaller than 50 milliequivalent/100 g, wherein a suspension formed by dispersing the clay particles in the deionized water at a concentration of 5 (w/v)% has a pH (25° C.) of not larger than 7.0, and has a proton emission capacity EH calculated according to the following formula, EH=(B-A)×10⁴ wherein A is a hydrogen ion concentration (g ions/L) of the above suspension, and B is a hydrogen ion concentration (g ions/L) of a suspension formed by dispersing the clay particles in a 1 wt% saline solution at a concentration of 5 (w/v)%, of not smaller than 0.5.

### BACKGROUND OF THE INVENTION

1. Field of the Invention

This invention relates to an orally administered adsorbent having excellent property for adsorbing nitrogen-containing compounds. More specifically, the invention relates to an orally administered adsorbent which, when orally administered, is capable of effectively adsorbing and removing nitrogen-containing compounds that accumulate in the body such as of human body or livestock when their kidney function has deteriorated.

2. Description of the Related Art

Excrements of livestock such as pigs, chicken, cows and sheep, pets such as dogs and cats (hereinafter called livestocks) and of humans, contain nitrogen-containing compounds such as urea, creatinine and uric acid as products of protein metabolism. The nitrogen-containing compounds are evacuated by the filtration/separation function of the kidney to avoid toxic substances and undesired substances from accumulating in the body as well as to adjust the body fluid osmotic pressure and the acid-base equilibrium. If the kidney function deteriorates, therefore, the above nitrogen-containing compounds accumulates in the bodies causing troubles in the living body such as uremia and trouble in the consciousness.

Among the nitrogen-containing compounds, further, the creatinine is contained in nearly a constant amount in the urine and serves as an effective index substance to represent the kidney function, and its serum creatinine value has been measured.

Active carbon is a known agent capable of removing the nitrogen-containing compounds that accumulate in the living body in case the kidney function has deteriorated. A patent document 1 is proposing a creatinine adsorbent obtained by coating active carbon with a compound having a group COOH or a group of a salt thereof.

[Patent document 1] JP-A-62-112564

### DISCLOSURE OF THE INVENTION

### Problems to be Solved by the Invention

The active carbon-type adsorbent disclosed in the patent document 1, however, is chiefly used for the dialysis and is not suited for being orally administered. The adsorbent of this kind is in the form of a black powder which lacks visual appeal and tends to cause such a trouble as constipation. Besides, its adsorptive ability is not sufficient yet.

In the case of a patient or animal such as dog, cat, etc. suffering from an advanced kidney failure, it is desired that toxic components such as nitrogen-containing compounds (creatinine, BUN, etc.) in the blood that were not excreted as urine be partly adsorbed through the mucous membrane of intestinal tracts and be excreted out of the body together with the evacuation.

It is therefore an object of the present invention to provide an orally administered adsorbent which, when orally administered, is capable of effectively adsorbing and removing nitrogen-containing compounds such as creatinine and the like.

### MEANS FOR SOLVING PROBLEMS

According to the present invention, there is provided an orally administered adsorbent comprising clay particles of a layered structure having a cation-exchange capacity of not smaller than 50 milliequivalent/100 g, wherein a suspension formed by dispersing the clay particles in the deionized water at a concentration of 5 (w/v)% has a pH (25° C.) of not larger than 7.0, and has a proton emission capacity EH calculated according to the following formula,
E_{H}=(B−A)×10⁴

wherein A is a hydrogen ion concentration (g ions/L) of the above suspension, and B is a hydrogen ion concentration (g ions/L) of a suspension formed by dispersing the clay particles in a 1 wt% saline solution at a concentration of 5 (w/v)%, of not smaller than 0.5.

In the present invention, it is desired that:
(1) The clay particles have a volume average particle size (D₅₀) of 3 to 100 μm as measured by a laser diffraction method;
(2) The clay particles are those of acid clay;
(3) An edible organic acid is contained in an amount of 0.01 to 20 parts by weight per 100 parts by weight of the clay particles; and
(4) Molded articles of the clay particles have a long diameter in a range of 0.1 to 10.0 mm, and a ratio of long diameter/short diameter of 1 to 10.

### EFFECTS OF THE INVENTION

As will be described in Examples appearing later, the orally administered adsorbent of the present invention adsorbs nitrogen-containing compounds and, particularly, creatinine and uric acid to a degree very higher than that of active carbon, and is very useful as a therapeutic drug or a preventive drug against the disease of decreased kidney function. The clay particles constituting the adsorbent is basically a natural aluminosilicate having hydrophilic property, and has been approved to be used as an additive for foods. Therefore, this adsorbent can be effectively used not only for livestocks but also for human bodies without causing such a trouble as constipation. In particular, the adsorbent is useful as an additive for foods for animals. The orally administered adsorbent of the invention further excellently adsorbs uric acid and can, hence, be used as a drug for preventing, for example, gout and for the therapeutic technique.

### PREFERABLY EMBODIMENT OF THE INVENTION

The clay particles constituting the adsorbent of the present invention have a layered structure as represented by a clay mineral of the group of, for example, montmorillonite having a cation-exchange capacity of not smaller than 50 milliequivalent/100 g, wherein a suspension formed by dispersing the clay particles in the deionized water at a concentration of 5 (w/v)% has a pH (25° C.) of not larger than 7.0 and, preferably, not larger than 5.0, and has a proton emission capacity EH of not smaller than 0.5 and, preferably, not smaller than 2.0. The layered clay mineral having the above characteristics not only excellently adsorbs the above nitrogen-containing compounds and, particularly, creatinine and uric acid but also is hydrophilic by itself without causing such an inconvenience as constipation when it is orally administered. Here, the proton emission capacity EH is calculated according to the following formula,
E_{H}=(B−A)×10⁴
wherein A is a hydrogen ion concentration (g ions/L) of the above suspension, and B is a hydrogen ion concentration (g ions/L) of a suspension formed by dispersing the clay particles in a 1 wt% saline solution at a concentration of 5 (w/v)%.

That is, the layered clay mineral as represented by montmorillonite basically has a three-layer structure of an SiO₄ tetrahedral layer-AlO₆ octahedral layer-SiO₄ tetrahedral layer, or a three-layer structure in which the above tetrahedral layers and the octahedral layer are substituted with different metals in the same manner permitting water and cations to be present among the laminated layers. Depending upon the substituent metals, elements among the layers, the kinds and quantities thereof, the layered clay mineral exhibits the above cation-exchange capacity, pH value and proton emission capacity E_{H}, and excellently adsorbs nitrogen-containing compounds.

Among many kinds of montmorillonite, the acid clay has a chemical structure in which Al atoms of the AlO₆ octahedral layer in the basic three-layer structure are partly substituted with a metal such as Mg or Fe(II), and hydrogen ions, calcium ions and sodium ions are bonded among the layers so as to compensate for an atomic value. Among them, the present invention uses the one that has the cation-exchange capacity, pH value and proton emission capacity E_{H} lying within the above ranges as an orally administered adsorbent.

Representative examples of the acid clay having the above properties are those having molar compositions lying in the following ranges on the basis of oxide moles, wherein R represents alkali metal components and M represents alkaline earth metal components:

R₂₀/SiO₂=0.1×10−2 to 1.5×10−2 (particularly, Na₂₀/SiO₂=0.3 ×10−2 to 1.0×10−2) and M₂₀/SiO₂=4.5×10−2 to 10.5×10−2

It is desired that the acid clay used in the present invention has a BET specific surface area of not smaller than 50 m²/g and an average porous diameter of 30 to 150Å.

The above layered clay mineral such as acid clay (acidic terra abla) is a natural aluminosilicate which has been approved as an additive for foods, contains MgO and CaO little due to its chemical composition, and permits such components to be little extracted with gastric acid. Therefore, the layered clay mineral little becomes an Mg source or a Ca source for the struvite urinary calculus, offering an advantage of exhibiting stable adsorbing action in the intestine.

Bentonite is a clay mineral pertaining to montmorillonite and has been orally administered being mixed into the feeds of livestocks. In the bentonite, however, ions present among the layers are almost all Na ions, and the proton emission capacity E_{H} is considerably lower than the above-mentioned range, and the pH value is as considerably high as 9.5 or more. As a result, the bentonite adsorbs the nitrogen-containing compounds to a degree considerably lower than that of the adsorbent of the present invention. Namely, the bentonite is used as a gastric antacid which is not suited for use as an adsorbent for nitrogen-containing compounds. Therefore, when the bentonite is used, the pH value must be adjusted to be not higher than 7.0 and, desirably, not higher than 5.0 by the treatment with an acid to a degree that does not destroy the layered structure, so that the proton emission capacity EH lies within the above-mentioned range.

As for the acid clay, as required, the pH value can be lowered to be not higher than 5.0 by the treatment with an acid.

The acid that is used may be an inorganic acid or an organic acid. Concretely, there can be used sulfuric acid, hydrochloric acid, nitric acid, citric acid or tartaric acid.

For example, the acid treatment of the acid clay was carried out by adding 0.05 N of HCl solution and stirring for one hour at room temperature, followed by filtration, washing and drying.

The acid clay of the present invention adsorbs part of toxic components in the blood accumulated in the body due to the decreased kidney function through the mucous membrane of intestinal tract, and accelerates the excretion out of the body, and is particularly effective for creatinine and uric acid. It is expected that lowering the toxic components in the blood helps improve clinical impressions such as poor appetite and languor. Concerning the reason for lowering the toxic components in the blood, it is considered that a common point is shared by the creatinine and by the uric acid which are the nitrogen-containing compounds capable of exhibiting keto-enol tautomerism as represented by the following formula (1) in the case of the creatinine and by the following formula (2) in the case of the uric acid. As will be learned from Examples 1, 4 and 5 appearing later, the creatinine and the uric acid are adsorbed both by 100%.

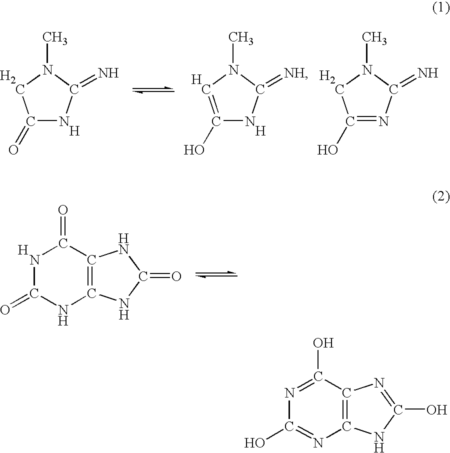

It is further desired that an edible organic acid is contained in an amount of 0.01 to 20 parts by weight and, preferably, 0.1 to 10 parts by weight per 100 parts by weight of the clay particles.

As the edible organic acid, it is desired to use carboxylic acid or oxycarboxylic acid that is permitted to be used as an additive for foods. Concretely, there can be used glacial acetic acid, propionic acid, butyric acid, benzoic acid, oxalic acid, succinic acid, adipic acid, lactic acid, malic acid, citric acid, gluconic acid or fumaric acid.

As required, further, the above edible organic acid salt can be used. As the salt, there can be used salts of potassium, sodium or ammonium.

By also containing the above edible organic acid or a salt thereof, it can be expected to obtain the effect for treating or preventing the urolithiasis.

According to the present invention, further, it is desired that the above clay particles have an average particle diameter (D₅₀) in a range of 3 to 100 μm as measured by the laser diffraction method. This is because when the molded articles of acid clay are dried at a temperature of, for example, not higher than 300° C., the molded articles collapse or swell in water or in an aqueous solution. When the particle diameter is larger than 100 μm, therefore, the effect of adsorption decreases. When the particle diameter is smaller than 3 μm, on the other hand, the acid clay adheres on the walls of intestine and the effect of excretion decreases.

For being orally administered to livestocks and men, it is desired that the molded articles of the clay particles have a long diameter in a range of 0.1 to 10.0 mm, and a ratio of long diameter/short diameter of 1 to 10 and, preferably, 1 to 3. The molded articles may be used in their own form or being mixed with other drug or feed.

The orally administered adsorbent of the present invention comprising the above clay particles is orally administered by itself into livestocks or men. To enhance the antacidic property, however, the orally administered adsorbent may be orally administered together with calcium carbonate. That is, the clay particles have a pH value that is shifted toward the acidic side and lose antacidic property, making a great difference from the bentonite. In the stomach, however, the calcium carbonate foams and decomposes upon reacting with acid, and produces a very large antacidic power to markedly improve antacidic property that is lost in the orally administered adsorbent of the present invention.

Usually, it is desired that the calcium carbonate is used in an amount of 5 to 50 parts by weight per 100 parts by weight of the clay particles. When the calcium carbonate is used in an amount greater than the above range, the antacidic property can be enhanced. To maintain adsorption for the nitrogen-containing compounds, however, it becomes necessary to use clay particles in large amounts. When the calcium carbonate is used in an amount smaller than the above range, the effect for improving the antacidic property by the calcium carbonate may become small.

The above calcium carbonate has no particular limitation on its particle size so far as it effectively foams and is decomposed with acid in the stomach, and can be used being mixed with the above clay particles in the form of a powder or particles of a particle size suited for being orally administered. In granulating the clay particles, further, the calcium carbonate may be mixed into the interior thereof upon the mixing and kneading. Then, the clay particles themselves collapse quickly due to the foaming and decomposition by the action of gastric acid, and the action as the adsorbent is exhibited more effectively. If decomposed in the stomach, there does not occur such an inconvenience that the clay particles adhere onto the walls of intestine.

### EXAMPLES

The invention will now be described in detail by way of Examples. The testing methods conducted in Examples were as described below.

(1) Cation-Exchange Capacity

Measured in compliance with the Standard Testing Method by the Association of Japan Bentonite Industries.

(2) Proton Emission Capacity

75 Grams of de-ionized water was introduced into a 100-mL beaker, 5 g of a sample was added thereto, and the mixture was boiled for 5 minutes. After cooled, the whole amount of the suspension was transferred into a messcylinder with a plug together with a small amount of the deionized water. Thereafter, another small amount of deionized water was added thereto so that the total amount was 100 mL which was stirred and mixed well. After the suspension was left to stand still for 3 minutes, a pH value was measured and a hydrogen ion concentration [A](g ions/L) was found by calculation. Further, the same operation was repeated by using a 1% saline solution instead of using the deionized water, and the hydrogen ion concentration [B](g ions/L) in the 1% saline solution was calculated. A difference (B-A) between the thus found hydrogen ion concentrations (g ions/L) was multiplied by 10⁴ and was regarded to be a proton emission capacity,
E_{H}=(B−A)×10⁴
(3) Creatinine Adsorption Ratio

Measured in compliance with the Jaff's method as described below.

The sample of a predetermined amount was accurately weighed into a plastic centrifugal sedimentation tube (12 mL), 5 mL of a creatinine solution of a predetermined concentration (creatinine concentration: 10 mg/100 mL) was added thereto, mixed together at room temperature for 10 minutes, and the mixture was subjected to the centrifugal separation at 2500 rpm (r=85 mm) for 15 minutes. 2 Milliliters of the supernatant liquid was taken into a new centrifugal sedimentation tube (12 mL) and into which were added 1 mL of a picric acid solution (22 mmols/L) and 1 mL of sodium hydroxide solution (0.75 mols/L), and the mixture was stirred and mixed together, and was left to stand in a water vessel controlled at 30° C. for 20 minutes. The above solution was further subjected to the centrifugal separation at 2500 rpm for 10 minutes, and the supernatant liquid was used as a sample solution to measure the absorbency at 520 nm by using a spectrophotometer. The creatinine concentration [C](mg/100 mL) of the solution was calculated by using a calibration curve prepared in advance through the same operation, and the creatinine adsorption ratio (%) of the sample was found from the following formula, Creatinine adsorption ratio=(10−C)÷10×100=(10−C)×10

(4) Uric Acid Adsorption Ratio

Measured in accordance with the ultraviolet absorptiometric method in a manner as described below.

The sample of a predetermined amount was accurately weighed into a plastic centrifugal sedimentation tube (12 mL), 10 mL of a urea solution of a predetermined concentration (uric acid concentration: 5 mg/100 mL) was added thereto, mixed together at room temperature for 10 minutes, and the mixture was subjected to the centrifugal separation at 2500 rpm (r=85 mm) for 15 minutes. The supernatant liquid was used as a sample solution to measure the absorbency at 284 nm by using an ultraviolet spectrophotometer. The uric acid concentration [U](mg/100 mL) of the solution was calculated by using a calibration curve prepared in advance through the same operation, and the uric acid adsorption ratio (%) of the sample was found from the following formula, Uric acid adsorption ratio=[(5−U)÷5]×100=(5−U)×20

### EXAMPLES 1 to 4

Samples shown in Table 1 were roughly milled into about 10 mm, and were molded each in an amount of 1 kg into cylinders of 1 mm by using a fine disk peletter (manufactured by Fuji Paudal Co.). The cylinders were dried at 150° C. for 6 hours. The dried products were milled by using a speed mill (manufactured by Showa Engineering Co.) to obtain granular products.

The contents of Examples, cation-exchange capacities, proton emission capacities, creatinine adsorption ratios and uric acid adsorption ratios were as shown in Table 1.

### EXAMPLES 5 and 6

Powdery samples shown in Table 1 were introduced each in an amount of 1 kg into a 10-liter plastic container, and to which 5 liters of a 0.05 N HCl solution was added and mixed at room temperature for 1 hour, followed by filtration and washing with 20 liters of clean water. The products were dried until the water contents were about 30%, and were molded into cylinders of 1 mm by using the fine disk peletter (manufactured by Fuji Paudal Co.). The cylinders were dried at 150° C. for 6 hours. The dried products were milled by using the speed mill (manufactured by Showa Engineering Co.) to obtain granular products.

The contents of Examples, cation-exchange capacities, proton emission capacities, creatinine adsorption ratios and uric acid adsorption ratios were as shown in Table 1.

### COMPARATIVE EXAMPLES 1 to 5

Powdery products of the samples shown in Table 1 were used.

The contents of Comparative Examples, cation-exchange capacities, proton emission capacities, creatinine adsorption ratios and uric acid adsorption ratios were as shown in Table 2.

### COMPARATIVE EXAMPLE 6

A powdery sample was introduced in an amount of 100 g into a 1-liter beaker, and to which 500 milliliters of a 0.05 N HCl solution was added and mixed at room temperature for 1 hour, followed by filtration and washing with 2 liters of clean water. The product was dried at 150° C. for 6 hours, and was milled in a mortar to obtain a powder thereof.

The contents, cation-exchange capacities, proton emission capacities, creatinine adsorption ratios and uric acid adsorption ratios were as shown in Table 2.

TABLE 1

| | | Amount | | | | | |
|---|---|---|---|---|---|---|---|
| | | of | | Cation-exchange | Proton | Creatinine | Uric acid |
| | | sample | | capacity (milli- | emission | adsorption | adsorption |
| | Sample name | (mg) | pH | equivalent/100 g) | capacity | ratio (%) | ratio (%) |
| Example 1 | acid clay 1*¹ sampled | 100 | 4.8 | 75 | 38 | 100 | 100 |
| | at Odo, Shibata-shi, | | | | | | |
| | Niigata-ken | | | | | | |
| Example 2 | acid clay 2 sampled | 100 | 5.4 | 56 | 2.6 | 98 | 75 |
| | at Odo, Shibata-shi, | | | | | | |
| | Niigata-ken | | | | | | |
| Example 3 | acid clay 3 sampled | 100 | 7.0 | 68 | 0.55 | 87 | 59 |
| | at Odo, Shibata-shi, | | | | | | |
| | Niigata-ken | | | | | | |
| Example 4 | acid clay sampled at | 100 | 4.8 | 88 | 39 | 100 | 100 |
| | Akatani, Shibata-shi, | | | | | | |
| | Niigata-ken | | | | | | |
| Example 5 | acid clay treated | 100 | 5.0 | 54 | 7.7 | 100 | 100 |
| | with an acid | | | | | | |
| Example 6 | natural bentonite | 100 | 6.0 | 79 | 3.6 | 78 | 56 |
| | treated with an acid | | | | | | |

*¹Acid clays 1 to 4 were sampled at different places in Odo.

TABLE 2

| | | Amount | | | | | |
|---|---|---|---|---|---|---|---|
| | | of | | Cation-exchange | Proton | Creatinine | Uric acid |
| | | sample | | capacity (milli- | emission | adsorption | adsorption |
| | Sample name | (mg) | pH | equivalent/100 g) | capacity | ratio (%) | ratio (%) |
| Comp. | acid clay powder 4 sampled | 100 | 9.2 | 81 | 1.0 × 10⁻⁴ | 5 | 0 |
| Ex. 1 | at Odo, Shibata-shi, | | | | | | |
| | Niigata-ken | | | | | | |
| Comp. | bentonite powder modified | 100 | 9.6 | 89 | 6.5 × 10⁻⁸ | 5 | 0 |
| Ex. 2 | with sodium carbonate | | | | | | |
| Comp. | sepiolite powder sampled | 100 | 9.0 | 27 | 1.0 × 10⁻⁵ | 9 | 0 |
| Ex. 3 | in Spain | | | | | | |
| Comp. | attapulgite powder sampled | 100 | 9.1 | 21 | 1.5 × 10⁻⁶ | 0 | 0 |
| Ex. 4 | in U.S.A. | | | | | | |
| Comp. | natural clinoptilite | 100 | 9.5 | 148 | 3.9 × 10⁻³ | 5 | 3 |
| Ex. 5 | powder | | | | | | |
| Comp. | sepiolite powder sampled | 100 | 7.3 | — | 6.5 × 10⁻⁷ | 13 | 2 |
| Ex. 6 | in Spain and treated with | | | | | | |
| | an acid | | | | | | |

*1: Acid clays 1 to 4 were sampled at different places in Odo.

1. An orally administered adsorbent comprising clay particles of a layered structure having a cation-exchange capacity of not smaller than 50 milliequivalent/100 g, wherein a suspension formed by dispersing said clay particles in the deionized water at a concentration of 5 (w/v)% has a pH (25° C.) of not larger than 7.0, and has a proton emission capacity E_{H} calculated according to the following formula,
E_{H}=(B−A)×10⁴
wherein A is a hydrogen ion concentration (g ions/L) of said suspension, and B is a hydrogen ion concentration (g ions/L) of a suspension formed by dispersing said clay particles in a 1 wt% saline solution at a concentration of 5 (w/v) %, of not smaller than 0.5.

2. The orally administered adsorbent according to claim 1, wherein said clay particles have a volume average particle size (D₅₀) of 3 to 100 μm as measured by a laser diffraction method.

3. The orally administered adsorbent according to claim 1, wherein said clay particles are those of acid clay.

4. The orally administered adsorbent according to claim 1, wherein an edible organic acid is contained in an amount of 0.01 to 20 parts by weight per 100 parts by weight of said clay particles.

5. The orally administered adsorbent according to claim 1, comprising molded articles of the clay particles having a long diameter in a range of 0.1 to 10.0 mm, and a ratio of long diameter/short diameter of 1 to 10.
